(19) United States
(12) Patent Application Publication
(10) Pub. No.: US 2024/0066166 A1
(43) Pub. Date: Feb. 29, 2024

(54) TREATMENT SYSTEM AND METHODS FOR ENCLOSED CHAMBERS

(71) Applicant: GCMG COMPANIES, LLC, Oviedo, FL (US)

(72) Inventors: Steven T. Grinstead, Oviedo, FL (US); George Nathen Pedro, JR., Christmas, FL (US)

(21) Appl. No.: 18/506,209

(22) Filed: Nov. 10, 2023

Related U.S. Application Data
(63) Continuation-in-part of application No. 17/657,804, filed on Apr. 4, 2022, now Pat. No. 11,819,586.
Application No. 17/657,804 is a continuation of application No. 16/991,180, filed on Aug. 12, 2020, now Pat. No. 11,291,740.
(60) Provisional application No. 62/885,414, filed on Aug. 12, 2019.
(60) Provisional application No. 63/488,201, filed on Mar. 3, 2023.
(60) Provisional application No. 63/497,839, filed on Apr. 24, 2023.
(60) Provisional application No. 63/480,408, filed on Jan. 18, 2023.

(51) Int. Cl. A61L 2/22; A61L 9/14

(52) U.S. Cl. CPC A61L 2/22; A61L 9/14

(57) ABSTRACT

A system for disinfecting an enclosed chamber may include a housing, an inlet and an outlet carried by the housing to be connected in an airflow path with the enclosed chamber, and a blower carried by the housing and connected to the airflow path between the inlet and outlet and configured to circulate air through the airflow path. The system may further include a disinfectant fog generator configured to introduce a disinfectant fog into the airflow path at room temperature from a disinfectant fluid, and a controller configured to operate the disinfectant fog generator and a dehumidifier coupled to the airflow path during a treatment phase.

### CROSS-REFERENCE TO RELATED APPLICATIONS

The present application is a continuation-in-part of U.S. application Ser. No. 17/657,804 filed Apr. 4, 2022, which is a continuation of U.S. application Ser. No. 16/991,180 filed Aug. 12, 2020, which claims the benefit of U.S. Provisional App. No. 62/885,414 filed Aug. 12, 2019, and it also claims the benefit of U.S. provisional app. Nos. 63/488,201 filed Mar. 3, 2023; 63/497,839 filed Apr. 24, 1923; and 63/480,408 filed Jan. 18, 1923, all of which are hereby incorporated herein in their entireties by reference.

### TECHNICAL FIELD

The present invention relates to the field of disinfecting, deodorizing, preserving, or sterilizing, and, more particularly, to apparatuses and methods for delivery of disinfecting, deodorizing, preserving, or sterilizing solutions.

### BACKGROUND

Risk factors associated with pathogen exposure from chambers and enclosures designed to be germ free are well known. Therefore, biological decontamination equipment is used to maintain the safety of these enclosures. Traditionally, specialized service providers are required to operate such equipment due to the inherent hazards in exposure to high concentrations of chemical sterilant in vapor or gaseous form.

For example, one approach that is used for sterilization purposes for such enclosures or other closed loop decontamination applications is decontamination by hydrogen peroxide (DHP). More particularly, a high concentration aqueous hydrogen peroxide solution (typically 50% or more by weight of H₂O₂) is evaporated, brought into contact with a hot gas stream and fed into the enclosure to be sterilized. This process is often called “gassing”. Afterwards, the enclosure is purged with air until the hydrogen peroxide level is at an approved safety level (e.g., 1 part per million by volume).

Nevertheless, such DHP delivery systems may pose safety risks not only in terms of the high concentrations of hydrogen peroxide used, but also as a result of the heating process. That is, typical approaches which rely on delivering high concentrations of H₂O₂ (or ClO₂) gas to enclosed spaces heat the gas so that it does not reach dew point levels. This is because concentrations of these solutions increase substantially on surfaces in which it condenses, causing significant material compatibility concerns and also concerns over accidental exposure to people from leaking chambers.

As a result, further advancements in decontamination equipment may be desirable in various applications.

### SUMMARY

A system for disinfecting an enclosed chamber may include a housing, an inlet and an outlet carried by the housing to be connected in an airflow path with the enclosed chamber, and a blower carried by the housing and connected to the airflow path between the inlet and outlet and configured to circulate air through the airflow path. The system may further include a disinfectant fog generator configured to introduce a disinfectant fog into the airflow path at room temperature from a disinfectant fluid, and a controller configured to operate the disinfectant fog generator and a dehumidifier coupled to the airflow path during a treatment phase.

In an example embodiment, the disinfectant fog generator may comprise an ultrasonic nebulizer. In accordance with another example embodiment, the disinfectant fog generator may comprise a vibrating mesh nebulizer. In still another example embodiment, the disinfectant fog generator may include an absorbent medium in the airflow path downstream from the blower, and a disinfectant fluid dispenser configured to dispense fluid to the absorbent medium responsive to the controller. In yet another example embodiment, the disinfectant fog generator may comprise an atomizing nozzle.

In some implementations, the controller may be configured to operate the blower non-continuously during the treatment phase. Also, in some example embodiments the controller may be configured to operate the disinfectant fog generator continuously during a first portion of the treatment phase, and non-continuously during a second portion of the treatment phase. By way of example, the dehumidifier may comprise a desiccation cartridge, and the system may further include an airflow valve downstream from the inlet and configured to switch the airflow through the desiccation cartridge responsive to the controller. In some embodiments, the dehumidifier may comprise an evaporator.

A related method for disinfecting an enclosed chamber may include connecting an inlet and an outlet of a fluid injection station in a closed airflow path with the enclosed chamber, the fluid injection station including a housing carrying the inlet and outlet, a blower carried by the housing and connected to the airflow path between the inlet and outlet, and a disinfectant fog generator coupled to the airflow path. The method may include operating the blower to circulate air through the airflow path during a treatment phase, operating the disinfectant fog generator to introduce a disinfectant fog into the airflow path at room temperature from a disinfectant fluid, and operating a dehumidifier coupled to the airflow path during the treatment phase.

### BRIEF DESCRIPTION OF THE DRAWINGS

FIG. 1 is a schematic block diagram of a system for disinfecting an enclosed chamber in accordance with an example embodiment.

FIG. 2 is a perspective view of an example fogging injection station which may be used in the system of FIG. 1.

FIG. 3 is a top view of the control panel of the fogging injection station of FIG. 2 shown in greater detail.

FIGS. 4-7 are display views of a touchscreen control panel which may be used with the fogging injection station of the system of FIG. 1 shown at various operational states of the fogging injection station.

FIG. 8 is a schematic block diagram of an alternative embodiment of the system of FIG. 1.

FIG. 9 is a flow diagram illustrating method aspects associated with the systems of FIGS. 1 and 8.

FIGS. 10A and 10B are a flow diagram of an example treatment method for an enclosed chamber which may be performed by the fogging injection stations of FIG. 2 or 8.

FIGS. 11-13 are schematic block diagram of other alternative embodiments of the system of FIG. 1.

### DETAILED DESCRIPTION

The present disclosure is provided with reference to the accompanying drawings, in which various embodiments are shown. However, other embodiments in many different forms may be used, and the disclosure should not be construed as limited to the particular embodiments set forth herein. Rather, these embodiments are provided so that this disclosure will be thorough and complete, and will fully convey the claim scope to those skilled in the art. Like numbers refer to like elements throughout, and prime notation is used to indicate similar elements in different embodiments.

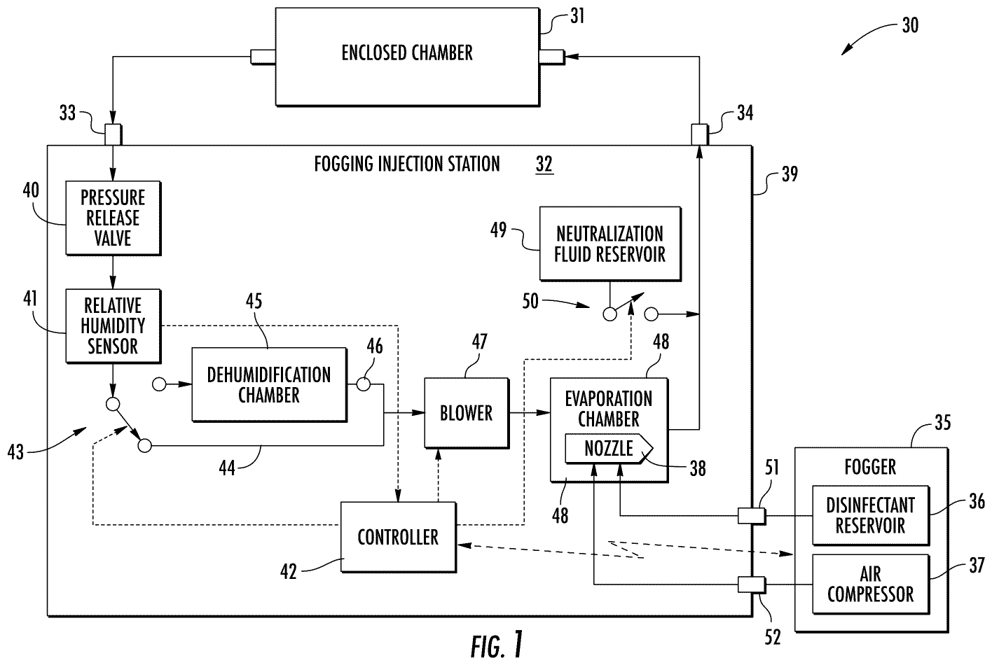

Referring initially to FIGS. 1-3, a system 30 for disinfecting an enclosed chamber or enclosure 31 illustratively includes a fogging injection station 32 which may be used to inject an atomized and/or vaporized fogging fluid into the enclosed chamber for the purpose of disinfecting and/or sterilizing the enclosed chamber. The fogging injection station 32 illustratively includes a housing 39, and an inlet 33 and an outlet 34 carried by the housing which are coupled to the enclosed chamber 31 to be treated. Examples of enclosed chambers 31 which may be treated using the fogging injection station 32 include gnotobiotic chambers, isolators, HEPA filter caissons for clean rooms, air handlers, biological safety cabinets (BSCs), animal transfer stations, hypoxia chambers, mobile laboratories, incubators, etc., and may be used for numerous applications including life sciences, pharmaceuticals, biomedical, and healthcare, for example. The system 30 advantageously provides for decontamination or sterilization of enclosed chambers to prevent human exposure to pathogens or other hazardous materials, as well as to provide disinfected/sterilized enclosures for medical or scientific applications, for example. It should be noted that in some embodiments, other types of fogging treatments may be performed by the fogging injection station 32, such as pesticide, deodorant, or preservative fogging, for example.

In particular, the fogging injection station 32 advantageously introduces an atomized disinfectant solution (e.g., an H₂O₂ solution) or fog into the closed airflow path between the fogging injection station and the enclosed chamber 31, which may provide desired efficacy with lower concentrations of H₂O₂, for example, and without the need for heated vaporization or boiling of the disinfectant fluid (e.g., at room temperature). That is, the fogging injection station 31 provides for an efficacious application under or over dew point without significant increase to material decomposition or accidental exposure due to lower parts per million concentrations of less than 200 PPM (and more particularly around 170 PPM), as compared to 400-800 or higher with prior approaches which provide a significantly greater risk to operators and those in proximity of the treatment area.

Beginning at the airflow inlet 33 and following the airflow path through the fogging injection station, an optional pressure release valve 40 may be incorporated in the airflow path to prevent the pressure within the airflow path from reaching a level or threshold which may damage components in the airflow path or the enclosed chamber 31. A relative humidity sensor 41 senses or measures the level of humidity in the airflow path, which is provided to and monitored by a controller 42. By way of example, the controller 42 may be implemented using a processor (e.g., microprocessor) and associated memory with non-transitory computer-readable instructions for causing the processor to perform the various operations described herein. Furthermore, a valve (e.g., a T-ball valve) 43 is downstream from the relative humidity sensor and, responsive to the controller 42, is configured to switch the airflow path between a bypass tube 44 and a dehumidification chamber 45. Another valve or de-coupler 46 allows the dehumidification chamber 45, along with the valve 43, to be completely shut off from the airflow path so that the dehumidification chamber may be serviced while the fogging injection station 32 is operational. For example, the dehumidification chamber 46 may be configured to receive a desiccant cartridge or pod, and the valves 43, 46 may be closed to allow a user to replace the desiccant pod as needed for humidity removal. However, in other configurations, the dehumidification chamber 46 may be used for an inline evaporator or dehumidifier, or the desiccant cartridge/dehumidifier may be external to the housing, for example. For the present discussion, a desiccation chamber is used. Moreover, in some embodiments the controller 42 may be remotely located from the housing 39 as well (e.g., in a separate human machine interface (HMI) used to control the fogging injection station 32, a cloud controller, etc.).

A blower or fan 47 is downstream from the desiccation chamber 46 and bypass tube 44, and its output blows through an evaporation chamber 48. The output of the evaporation chamber 48 is connected to the airflow outlet 34 of the fogging injection station 32. An optional neutralization fluid reservoir 49 may also be connected into the airflow path between the evaporation chamber 48 and outlet 34 (or directly into the evaporation chamber in some configurations) via a valve 50, which is controlled by the controller 42. It will be appreciated, however, that in different embodiments various components may be located in different positions along the airflow path. Operation of the above-noted components will be described further below.

In the illustrated example, the fogging injection station 32 is coupled to an atomizing fogging device or fogger 35, which includes a disinfectant reservoir 36 for the disinfectant (e.g., a H₂O₂ solution, etc.), and an air compressor 37, both of which are in fluid communication with an atomizing nozzle 38 in the fogging injection station which accordingly generates atomized disinfectant in the airflow pathway between the fogging injection station and the enclosed chamber 31. In some embodiments, a priming pump may be used to prime the atomizing nozzle 48 upon startup of the treatment cycle (or later, if appropriate). One particularly advantageously example of such a fogging device 35 is set forth in U.S. Pat. No. 10,092,668 to Grinstead, which is hereby incorporated herein in its entirety by reference.

The fogger 35 also includes its own atomizing nozzle (not shown) and may be used for the treatment of enclosed areas or rooms on its own, but then supply the air compressor 37 and disinfectant reservoir 36 for the fogging injection station 32 when an enclosed chamber 31 is to be treated. In this regard, the fogger 35 and fogging injection station 32 may be used to not only treat enclosed chambers 31, but also the rooms in which the enclosed chambers are present (e.g., as in a laboratory setting). In an example implementation, the housing 39 of the fogging injection station 32 may be a cart with a shelve or rack for the fogger 35, so that the fogger may be housed within and connected with the fogging injection station during treatment of the enclosed chamber 31, and then removed for treatment of the room or storage area where the enclosed chamber is located. Quick connect ports 51, 52 may be used for connecting the disinfectant reservoir 36 and air compressor 37, respectively, to the fogging injection station 32.

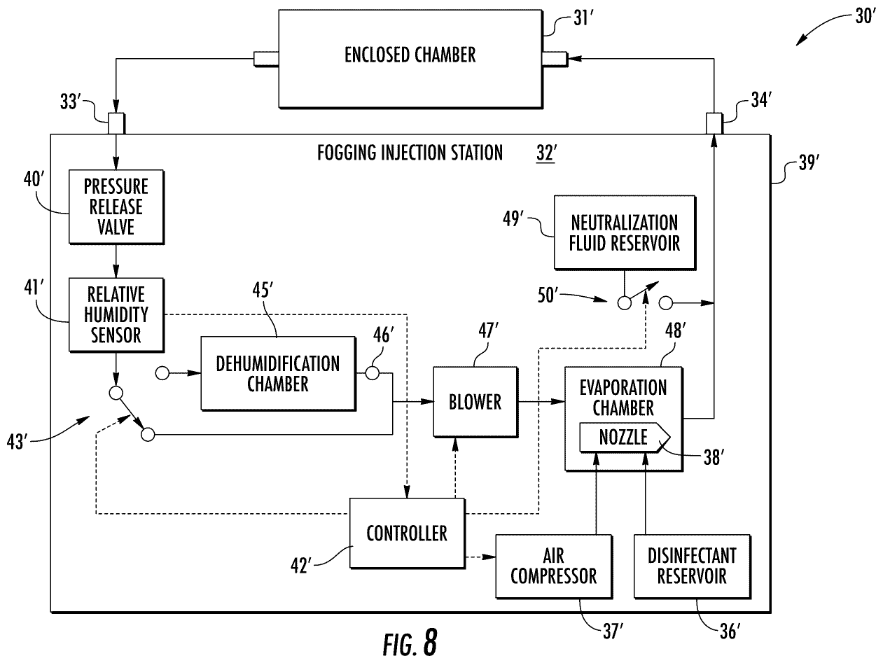

In an alternative embodiment of the fogging injection station 32′ shown in FIG. 8, various components of the atomizing fogging device 35 (e.g., air compressor 37′, fluid reservoir 36′, etc.) may be included in the fogging injection station itself such that a separate fogging device is not required. That is, the fogging injection station 32′ may be considered as an integrated or stand-alone fogging injection station that does not require a separate fogger to be connected thereto.

An example operation flow of the fogging injection station 32 is as follows. Air from the enclosed chamber 31 enters the air inlet 33 of the fogging injection station 32. From there it passes the optional pressure release valve 40 and comes across the relative humidity sensor 41, followed by the valve 43. In the case of a hydrogen peroxide disinfectant, the humidity sensor 41 may be a hydrogen peroxide sensor, or a separate hydrogen peroxide sensor may be included as a secondary type of humidity sensor to measure not only the general humidity in the airflow path, but the H₂O₂ in the airflow path as well. However, it should be noted that other types of disinfectants as well as sensors (e.g., temperature sensors, etc.) may be used to provide information to the controller 42 in different embodiments as well. Rigid and/or flex PVC tubing may be used to interconnect the components in the airflow path, and may generally be in a range of 1-3″ in diameter (e.g., 2″ tubing), although different types of tubing and dimensions may be used in different configurations.

The valve 43 is positioned in the airflow path to allow the airflow to be coupled with the attached fogging device 35 during a treatment phase (via the bypass tube 44 in the illustrated example), and to the dehumidification (e.g., desiccation) chamber 45 during desiccation phase to remove the disinfectant from the enclosure. A solenoid (not shown) may be provided to work in conjunction with the valve 43 for switching between the bypass (disinfecting) and desiccation phases. The controller 42 may be programmed to only allow air flow through the dehumidification chamber 45 during the desiccation phase.

The blower 47 is connected in the flow path and blows air through the evaporation chamber 48 and out through the outlet 34 of the fogging injection station 32 and back into the enclosed chamber 31. As noted above, the dehumidification chamber 45 may be a desiccant chamber for receiving a desiccant or silica gel. In one example, molecular sieve is used as the desiccant, although other suitable desiccants may be used in different embodiments. As noted above, the desiccant may be placed within a removable cartridge or pod, so that new cartridges may be swapped in and out of the chamber 45 as appropriate.

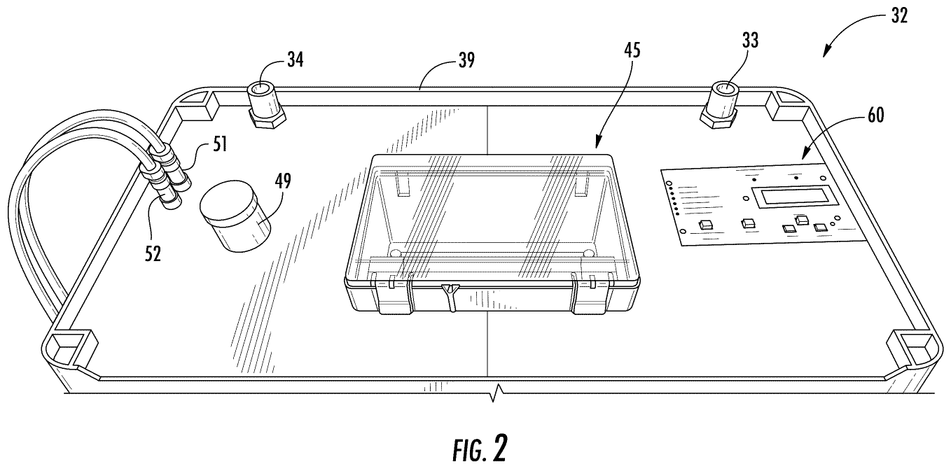

The atomizing nozzle 38 is connected to the disinfectant reservoir 36 and air compressor 37 of the fogger 35 by a pair of side discharge hoses, for example (see FIG. 2). The atomizing nozzle 38 is connected in line to the airflow path within the evaporation chamber 48. That is, the nozzle 38 points in the direction of air flow (downstream toward the outlet 34). When the blower 47 is turned on so that air is passing through it, and the controller 42 instructs the fogger 35 to operate the air compressor 37 (and optionally priming pump), the nozzle 38 sprays atomized disinfectant solution in line in the airflow path. However, in some embodiments the nozzle 38 may be oriented differently.

The atomizing nozzle 38 produces a hybrid aerosolized fogging disinfectant, in that some of the solution will be in a gaseous state (vapor) and some will be in a liquid (droplet) state. For enclosures that have HEPA filters, for example, such filters may prevent passage of the atomized liquid, but the vapor will more readily pass through the HEPA filter. However, the evaporation chamber 48 advantageously helps convert atomized liquid into vapor as well, so that it too may pass into the enclosed chamber 31.

The optional pressure relief valve 40 may also be included in the fogging injection station 32 in the event there is an unexpected rise in pressure. This advantageously helps protect enclosed chambers 31 such as gnotobiotic chambers, for example, that could potentially rupture if the internal pressure is increased too much, and potentially components in the fogging injection station 32 as well. The pressure relief valve 40 may be coupled in the airflow path at other locations prior to the outlet 34 in some embodiments.

In one example implementation, approximately sixteen cubic feet of air is moved through the evaporator per minute (although other amounts of air flow may be provided in different embodiments). If humidity is in a range of 30 to 50%, the vast majority of the humidity will be evaporated into the air flow that goes into the enclosure 31. Accordingly, the fogging injection station 32 is advantageously able to evaporate the suspended liquid droplets in the aerosolized fog injected from the nozzle 38 to advantageously pass through HEPA filters of the enclosed chamber 31.

When the treatment phase is complete, the fogging injection station 32 may then enter an extraction phase when it evacuates the vaporized solution from the enclosed chamber 31. During this phase, the controller 42 causes the solenoid and valve 43 to route the air flow through the desiccation chamber 45, which removes the humidity and, in the case of a hydrogen peroxide-based disinfecting solution, neutralizes and removes it from the treated chamber 31. The type of desiccant and quantity thereof may be selected to provide appropriate extraction for the size of the enclosed chamber 31 being treated and amount of solution that will be introduced into the chamber, as will be appreciated by those skilled in the art. In the example of FIG. 2, a lid on the top of the fogging injection station 32 allows for easy access into the desiccation chamber 45 to replace desiccation pods.

In some embodiments, a neutralization phase may also be provided in which a neutralizing fluid or aqueous solution is introduced into the airflow path from the neutralization fluid reservoir 49 after the extraction phase, such as deionized water, for example. The neutralization solution may advantageously help neutralize any acids remaining on materials and also neutralize ions left behind by the H₂O₂ process, which could otherwise degrade materials and/or cause health side effects. In the example of FIG. 2, access to the reservoir 49 is provided on the top of the fogging injection station 32 so that the neutralization solution may be easily refilled. The reservoir 49 may hold on the order of a few ounces of neutralization solution, although larger reservoirs may be used depending on the application. Moreover, in some implementations a pump (not shown) may be associated with the neutralization fluid reservoir 49 and controlled by the controller 42 to pump the neutralization solution into the airflow path during and/or after the evaporation phase. However, other configurations may also be possible (e.g., a gravity fed configuration).

To summarize the operational phases of the fogging injection station 32, as described in the above-noted '668 patent, the initial treatment or killing phase when the disinfectant is being introduced into the enclosed chamber 31 may be divided into two sub-phases, namely a continuous injection to initially bring the chamber up to the desired level (e.g., humidity or H₂O₂ level), and then a non-continuously (e.g., intermittent or periodic) injection to maintain the chamber at the desired level (or within a desired range). However, it should be noted that in other embodiments other injection configurations may be used (e.g., just continuous injection, etc.). By way of example, the treatment phase may typically range from thirty minutes to an hour, although other times may be used in different applications.

After the treatment phase, the fogging injection station 32 enters the extraction phase when the disinfectant (e.g., a hydrogen peroxide-based solution) is removed by the desiccant. The desiccation chamber 45 pulls out humidity in liquid form. Moreover, in the case of molecular sieve, for example, it also pulls out gas because it is porous enough to grab the gas molecules as well.

Then, an optional neutralization phase may be provided when the fogging injection station 32 introduces a neutralizing fluid (e.g., deionized water), which brings the humidity up in the enclosure. The hydrogen peroxide is mostly or completely evacuated out of the enclosed chamber 31 being treated by the time of the neutralization phase, which helps to ensure there are no undesired residues or ions within the enclosed chamber as noted above. The fogging injection station 32 may then run a second evaporation phase to lower the humidity in the enclosure (e.g., to remove the deionized water) after the neutralization phase, if desired. Another approach is to just vent or resume normal air flow to the enclosed chamber 31, since at this point it just has air and water (in the case of deionized water as the neutralization fluid) therein.

The controller 42 switches the actuators for the above-noted valves and pumps to connect the spray nozzle, evaporation chamber, desiccation chamber, and neutralization fluid reservoir in the airflow path during the appropriate operational phase. Moreover, in some embodiments, the controller 42 may also control the fogging device 35 (e.g., wirelessly or by a wired connection) to coordinate its operation during the treatment phase, as described further in the above-noted '668 patent. Furthermore, the controller 42 may also be used to control other foggers, e.g., within the same room the fogging injection station is being used in, to coordinate their treatment cycles as well, if desired, as also described further in the '668 patent. The controller 42 may also be configured to change operating parameters during the treatment cycle as appropriate. For example, if it is taking longer for an enclosure 31 to reach the desired humidity or H₂O₂ level (as measured by the in-line humidity or H₂O₂ sensor) than the baseline programming provides for, the controller 42 may cause the fogging device 35 to extend the continuous injection phase for a longer period of time before switching to the pulse (non-continuous) injection phase, for example.

More particularly, the various points at which the controller 42 switches between the treatment, evacuation, and neutralization phases may be based upon estimated times for the particular enclosed chamber 31 being treated, which are set through baseline programming. Another option is that switching may occur based upon relative humidity (or H₂O₂) levels in the airflow as measured by the relative humidity sensor 41 (and/or H₂O₂ sensor). Relative humidity set points may be provided as part of the baseline programming by the manufacturer, or in some embodiments an interface may be provided so that a user may adjust these values based upon the particular type of enclosed chamber 31 being treated, the environment in which the treatment is occurring, etc. In some embodiments, a combination of set times and set humidity levels may be used, such that the various phases run for the predetermined time unless completed earlier as measured by the humidity sensor 41 (and/or H₂O₂ sensor). In this regard, the controller 42 may also monitor the rate of change of the humidity level, during the evacuation phase, for example, and if the rate is slower than a threshold rate then the process may be stopped, and the user prompted to replace the desiccant pod.

In some embodiments, the controller 42 may also turn the blower 47 on and off during the pulsed (non-continuous) portion of the treatment phase. More particularly, it is generally desirable that the blower 47 is running any time disinfectant is being atomized in the airflow path, as this not only helps to circulate the atomized disinfectant to the enclosed chamber 31, but it also aids in the evaporation of the liquid disinfectant droplets as discussed above. However, the continuous air flow may in some enclosed chambers result in eddies or “dead zones” where less atomized disinfectant reaches. This may occur as a result of the particular geometry of the cabinet or enclosure being treated. Pulsing the air flow by cycling the blower 42 may advantageously help force more atomized disinfectant into these dead zones to help ensure that proper disinfection occurs uniformly throughout the enclosed chamber 47. By way of example, the controller 42 may cause the blower 47 to cycle on and remain on while the disinfectant is being pulsed during the second portion of the treatment phase, but cycle off briefly between disinfectant pulses (non-continuous operation).

As noted above, in some embodiments the separate fogger 35 (or fogging components, such as a compressor and atomizing nozzle) could be omitted. In a variation of the fogging injection station 32′, the treatment fluid/disinfectant reservoir 36′ may be connected directly to the evaporation chamber 48 without the air compressor 37′ or atomizing nozzle 38′, such that the disinfectant is evaporated directly into the airflow path without first being atomized. However, this may require a longer duration to bring the enclosed chamber 31′ up to the desired level of disinfectant or humidity, and thus an extended treatment cycle.

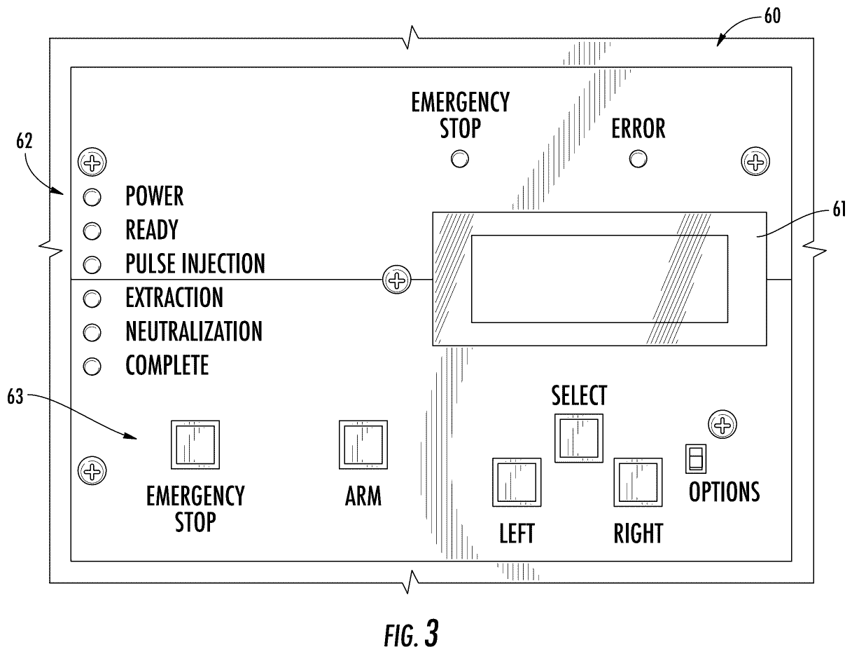

A control panel 60 of the fogging injection station 32 illustratively includes a display 61 and indicators 62 (e.g., LEDs, etc.) as output devices, as well as user input devices 63 (e.g., buttons, touch screen, etc.) which are coupled to the controller 42 to allow a user to initiate and monitor the treatment cycle and the various operational phases (see FIG. 3). In some embodiments, the fogging injection station 32 may allow for calibration prior to a treatment cycle or as desired to calibrate the various sensors therein. During calibration, the fogging injection station 32 is not connected to an enclosure (a bypass tube may optionally be connected between the input and output ports 33, 34), and the blower 47 runs to cycle air through the fogging injection station. Calibration may be appropriate to remove humidity from the fogging injection station 32 from a previous treatment cycle, etc. The fogging injection station 32 may also allow for a forced extraction during a treatment cycle to provide forced desiccation through the desiccation chamber 45, essentially functioning as a dehumidifier to remove humidity from the enclosure 31.

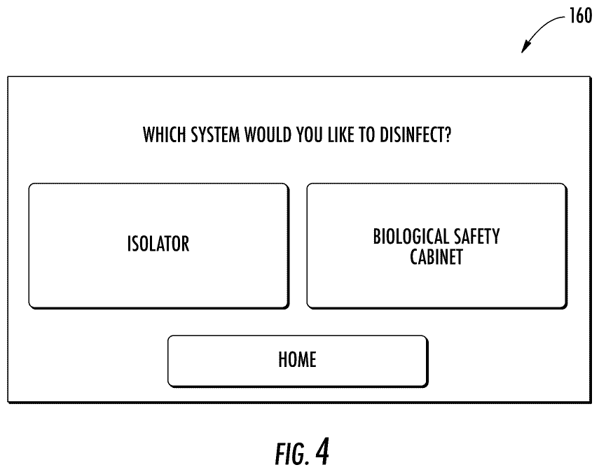

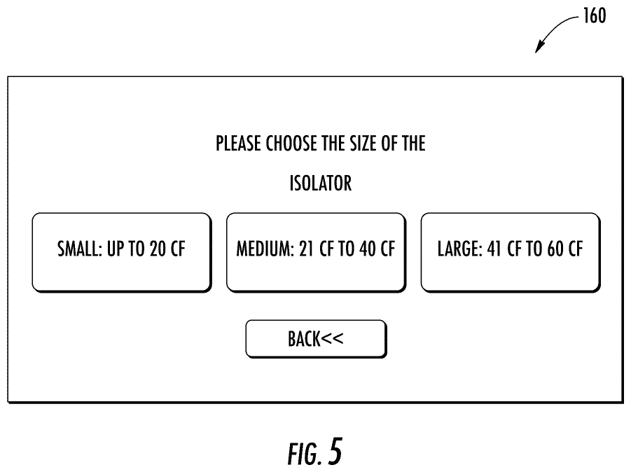

Another example control panel configuration is a touchscreen display 160 shown in FIGS. 4-7. In the example shown in FIGS. 4-5, the controller 42 may advantageously store different types of enclosed chambers 31 and associated parameters/settings. This makes it easier and quicker for users to configure the fogging injection station 32 for use with different types of enclosed chambers 31. In the illustrated example, the user is first asked what type of enclosed chamber 31 is being disinfected, and two options are provided for selection (see FIG. 4), namely isolators and biological safety cabinets (although other types of enclosures may be listed in different embodiments). Once one of the types of enclosed chambers 31 is selected, then further information regarding the chamber may be collected by the controller 42 to determine the appropriate treatment cycle settings.

In the illustrated example, the user has chosen to treat an isolator, and the user is accordingly prompted to enter a size of the isolator (FIG. 5). Here, the user is presented with three options, namely small, medium, and large isolator sizes. However, other approaches may be used, such as allowing the user to enter dimensions of the isolator directly, or entering a make/model of the isolator from which the controller 42 may retrieve previously stored parameters (dimensions, volume, etc.) to select the appropriate treatment cycle settings. In some implementations, the controller 42 may store these settings locally, or interface with a database (e.g., via Wi-Fi, etc.) so that these settings may be stored and accessed across different fogging injection stations 32, for example. In some embodiments, the fogging injection station 32 may be configured as a dedicated unit during a cycle development process to work with a specific type of enclosure, such that the operating parameters are fixed for the particular implementation (e.g., for regulatory compliance).

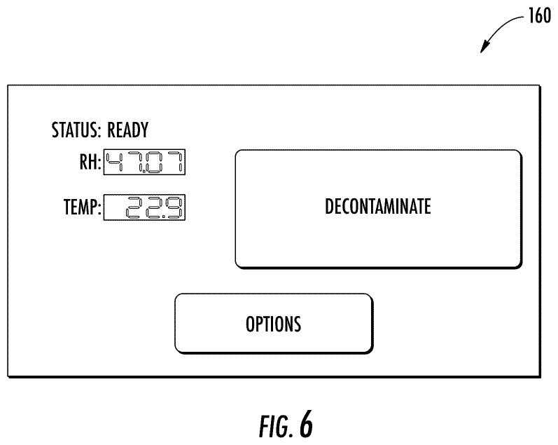

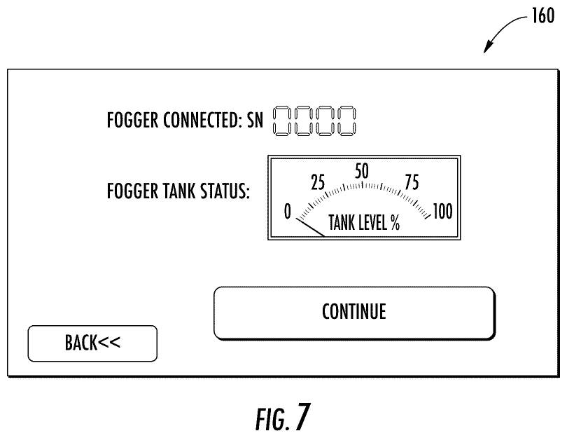

In the example of FIG. 6, current conditions are displayed, including the measured humidity and temperature of air within the airflow path. A “decontaminate” button allows for starting the treatment cycle, and an options button allows for the selection or customization of other treatment cycle settings and/or other settings of the fogging injection station 32 (e.g., Wi-Fi setup, display options, fogger pairing, etc.). Other status display settings may include a fogger tank status (e.g., disinfectant fill level) and paired fogger identification/status (see FIG. 7), although others may be provided as well.

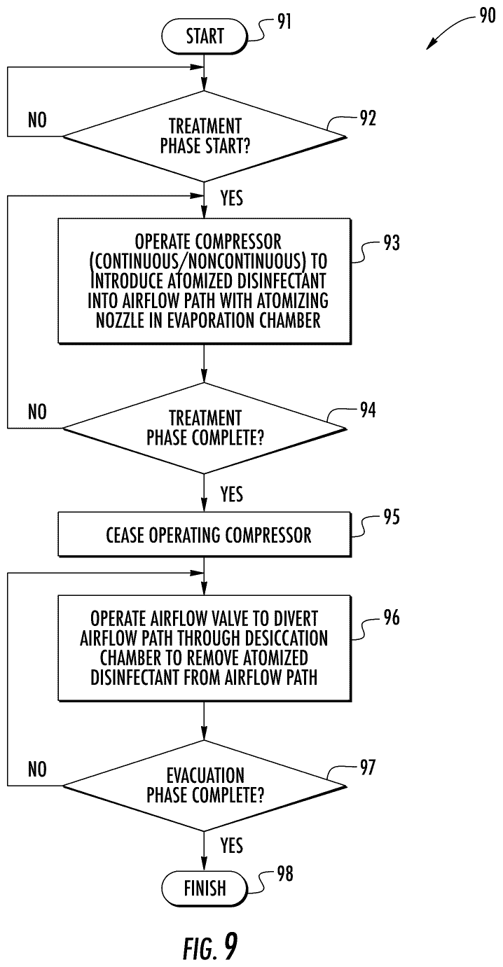

Turning now to the flow diagram 90 of FIG. 9, a related method for disinfecting an enclosed chamber 31 illustratively begins at Block 91. Upon initiation of a treatment phase (Block 92), the controller 42 operates the air compressor 37 to introduce atomized disinfectant into the airflow path, as discussed further above (Block 93). When the treatment phase is complete (Block 94), the evacuation phase begins with ceasing operation of the air compressor 95 to stop the atomized disinfectant injection, at Block 95, along with operating the solenoid and airflow valve 43 to divert the airflow path through the dehumidification chamber 45 to remove atomized disinfectant from the airflow path, at Block 96, as also discussed above. Upon completion of the evacuation phase (Block 97), the method of FIG. 9 illustratively concludes at Block 98. However, in some embodiments, the optional neutralization phase may be performed along with additional evacuation/dehumidification as desired.

The fogging injection station 32 set forth herein advantageously provides desired efficacy, yet with a delivery platform that helps mitigate the risks to both staff and facilities, and may help decrease overall hazards involved with the decontamination process. Moreover, this approach also allows for the decontamination of self-contained germ-free enclosures like gnotobiotic, isolators, hypoxia and biological safety cabinets without the use of relatively high concentration level disinfectants (e.g., 50% and higher H₂O₂ solutions). Rather, the present approach may utilize relatively low-level concentrations, e.g., in the 7-10% range (or potentially less). Moreover, relatively low PPM levels (e.g., below 200 PPM) may be achieved without heating to provide safer operation and less potential for damage to the enclosures.

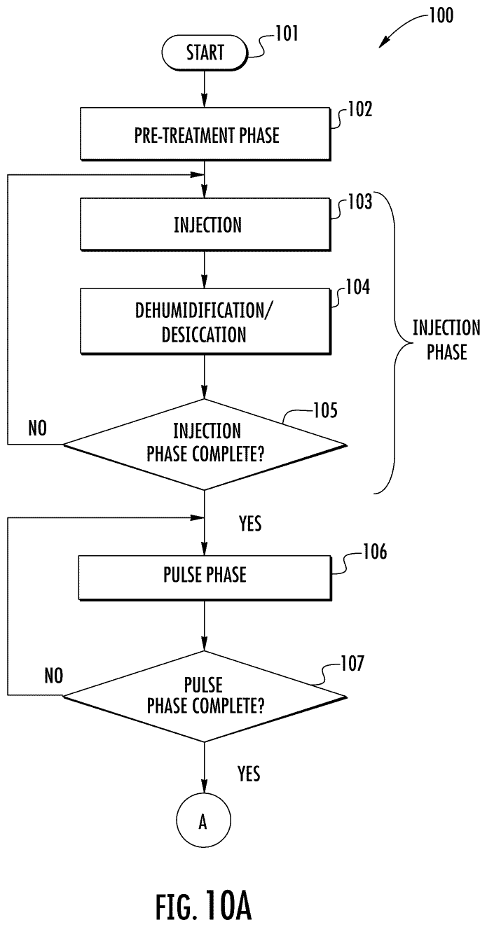

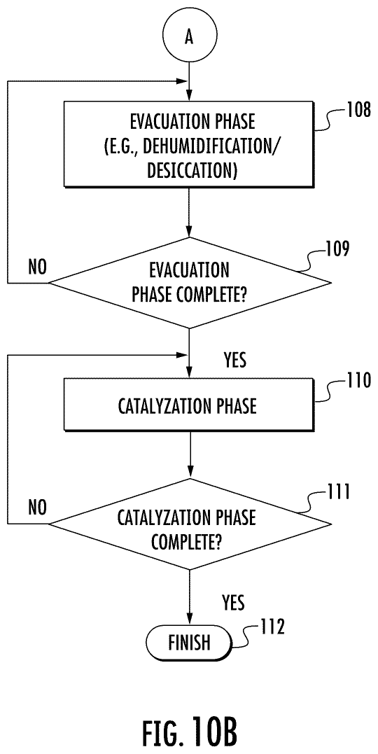

Turning to the flow diagram 100 of FIG. 10, an example treatment cycle which may be performed by the fogging injection station 32 (or other fogging devices in different embodiments) is now described. Beginning at Block 101, the treatment cycle may begin with an optional pre-treatment phase, at Block 102. By way of background, there may be extended periods of time between runnings of decontamination cycles in some instances, such as over a weekend when a laboratory or other facility is closed. In such cases, the active ingredient(s) in a treatment solution (e.g., H₂O₂) that remains in the nozzle 38 will be exposed to the metal in the nozzle over the extended period of time, resulting in catalyzation of the active ingredient(s). Over such an extended period, catalyzation will result in a reduction of the percentage of the active ingredient(s) in the treatment solution that remains in the nozzle will be reduced.

Though the amount of fluid in the nozzle 38 subject to catalyzation will be relatively small (e.g., on the order of a few milliliters), for a relatively small chamber 31 (e.g., a pass-through chamber for laboratories, etc.), the lowered concentration of the active ingredient(s) may have a significant effect, as compared to beginning the treatment cycle with fresh (i.e., full or nearly full strength) solution. That is, an injection phase for a small, enclosed chamber 31 may be relatively short given the small volume of such chambers, and thus beginning with treatment solution that has a reduced concentration of the active ingredient(s) may result in the chamber not reaching the target concentration during the treatment cycle.

To this end, during the pre-treatment phase the controller 42 may cause the dehumidification chamber 45 to be operated at the same time that atomized treatment solution is being injected into the enclosed chamber 31 via the nozzle 38 to rapidly draw down humidity in the chamber. That is, when a water-based treatment solution is used, a rapid draw down of humidity may cause an increase or spike in the percentage (parts per million) of an active ingredient such as H₂O₂ in the enclosed chamber 31. However, operating the dehumidification chamber 45 for too long may be counterproductive, in that it may begin to also extract the active ingredient (e.g., H₂O₂) and/or excessive amounts of the full-strength treatment solution from the enclosed chamber 31. By way of example, the time range for operating the dehumidification chamber 45 during a pre-treatment cycle may be on the order of several seconds, such as between five and sixty seconds, and more specifically in a range of 10 to 30 seconds, although other times may be used in different embodiments.

In this regard, performing the pre-treatment step helps to ensure that test results will be consistent and that desired target levels of the active decontamination ingredient in the enclosed chamber 31 will be reached. To this end, the controller 42 may keep track of the time between when the fogging injection station 32 is used. For example, if the fogging injection station 32 is used multiple times within a day, then there may be no need to perform the pre-treatment step between treatment cycles. However, if the fogging injection station 32 has been idle overnight, for example, then the controller 42 may cause the pre-treatment cycle to be performed during the first treatment cycle of the next day.

By way of example, a threshold delay period which may trigger use of the pre-treatment cycle may be in a range of three hours or more, and more particularly eight hours or more, though delays of shorter than three hours may also be used in some implementations. In other words, the controller 42 may track how long it has been since the last treatment cycle (e.g., measured from when the last cycle was completed, or when the compressor 37 was last turned off) and automatically initiate the pre-treatment cycle only when the threshold delay period (e.g., eight hours) has been exceeded since the prior treatment was performed. In some embodiments, users may also be able to manually select when the pre-treatment is to be performed (e.g., before every treatment cycle), or adjust the threshold delay period, e.g., through a user interface (UI), etc.

Once the pre-treatment phase is complete, an injection phase may commence, at Block 103. The purpose of the injection phase is to cause the enclosed chamber 31 to reach its target concentration of humidity/active ingredient. However, rather than continuously injecting treatment fluid for the entire injection period, the fluid injection is intermittently or periodically paused to allow dehumidification to be performed for brief periods, e.g., fifteen seconds to five minutes depending on the enclosed chamber 31 volume, although other durations may be used in different embodiments. As discussed further above, for a water-based treatment solution such as an aqueous H₂O₂ solution, relatively short periods of dehumidification or desiccation will cause the percentage (parts per million) of the H₂O₂ in the enclosed chamber 31 to more rapidly increase as measured by an H₂O₂ sensor compared to continuously injecting the atomized treatment solution via the nozzle 38 without any dehumidification/desiccation.

The above-described injection with intermittent or periodic dehumidification/desiccation provides a number of technical advantages. Applicant theorizes without wishing to be bound thereto that, for an aqueous treatment solution of a given concentration of active ingredient, the enclosed chamber 31 will reach a target concentration of the active ingredient quicker using this approach versus a continuous injection of the same treatment solution.

Another technical advantage is that this approach may allow for desired efficacy with aqueous treatment solutions having a lower concentration of the active ingredient. For example, an aqueous 3% H₂O₂ solution might not have a sufficient concentration of H₂O₂ to otherwise reach a target concentration using a continuous injection without oversaturating the enclosed chamber. However, the intermittent dehumidification/desiccation may cause the percentage of H₂O₂ in the enclosed chamber to increase more rapidly, which effectively makes the aqueous 3% H₂O₂ solution perform like a higher concentration H₂O₂ solution would with continuous injection. Stated alternatively, this approach may cause an aqueous disinfectant solution of a lower active ingredient concentration to perform like an aqueous solution of a higher active ingredient concentration would during a continuous injection. This may be beneficial in that a solution with lower concentration of an active ingredient(s) may be less caustic and therefore potentially subject to less restrictions or difficulties with regard to storage, transportation, and/or use.

The total time for the injection phase including intermittent or periodic dehumidification/desiccation will vary depending on the size or volume of the enclosed chamber 31. For example, a pass-through chamber may only require an injection phase of a few minutes or less to reach an initial desired target saturation, while a larger chamber such as a biological safety cabinet or a room will take a longer time for the injection phase (e.g., five to fifty minutes), although different times may be used in different applications.

As discussed above, the appropriate fogging time for the particular chamber 31 being treated may be determined by the controller 42 upon selection of a given chamber type by the user through the UI, for example. In some configurations, the injection phase duration may be manually set as well. It should also be noted that in some embodiments, rather than pausing the injection of atomized fluid during the dehumidification/desiccation intervals, the atomized fluid may be continuously injected as described above with respect to the pre-treatment phase. Similarly, during the pre-treatment phase, injection could be paused during the dehumidification/desiccation interval(s), if desired.

After the injection phase is complete (Block 105), an optional pulse phase may occur for the appropriate amount of time, at Block 106. In an example embodiment, for an aqueous 5% H₂O₂ treatment solution being injected into a large chamber or room, with a relative humidity saturation level of greater than 85%, and more particularly between 90 and 95%, an example pulse phase for this use case may be 45 seconds on, 15 seconds off during each minute of the pulse phase, although other cycle times and saturation levels may be used depending on the given application, treatment solution, and/or enclosed chamber 31 being treated.

Generally speaking, Applicant theorizes without wishing to be bound thereto that during the injection phase, enough chemical should be added to the enclosed area to bring the area to around 90% relative humidity or more for the above-described H₂O₂/H₂O mixture. More particularly, with such a chemical mixture, when the relative humidity is above 85% then the enclosed area may be considered to be in the “kill zone” where most if not all pathogens will be killed if exposed for a sufficient duration at this concentration. Thus, the amount of time necessary for the injection cycle may vary depending on the starting relative humidity, and additional time may be required where the starting humidity is relatively low, for example, to reach the kill zone. The purpose of the pulse phase is to keep the enclosed area in the kill zone.

In accordance with another example dwell cycle implementation, the pulse phase may be broken into five-minute programmable segments (although other durations may also be used). Each segment may include compressor 37 cycling on/off for a given time (e.g., 100 seconds off, 100 seconds on, 100 seconds off, although other durations may be used and the on/off times may be different). This ratio may change based upon the given fog or saturation time. That is, for a shorter fog time there may be a shorter ON segment, and a longer fog time may have a longer ON segment, for example. The length of the pulse time may advantageously be adjusted (e.g., in a range of 10 to 40 minutes, although longer or shorter times may be used depending upon the size of the area under treatment) based upon the particular pathogen(s) that is targeted. By way of example, a relatively short pulse phase of ten minutes may be sufficient for a relatively easy to kill pathogen, while a longer time (e.g., 25 minutes or more) may be used to kill more difficult pathogens.

Completion of the pulse phase is determined at Block 107, after which an optional evacuation phase may commence (Block 108). By way of example, this may include running the dehumidification (e.g., desiccation) chamber 45 for a time period, operating a separate dehumidifier, and/or venting the enclosed chamber 31, for example. Once the evacuation phase is complete (Block 109), then an optional catalyzation phase 111 may be performed. By way of example, in some embodiments the fogging injection station 32 may include a catalyzation chamber for receiving a catalyst pod, similar to the desiccation pod described above. That is, the pod includes a catalyst medium that may be used to help further break down the active ingredient that remains within the enclosed chamber 31.

For the above-described example of an H₂O₂ treatment solution, a manganese dioxide (MnO₂) filter medium may be particularly helpful to dissipate or neutralize the H₂O₂ in the enclosed chamber 31. That is, cycling air from the enclosed chamber 31 through the catalyst medium will help to more rapidly bring the concentration of H₂O₂ in the enclosed chamber to a level that is safe, allowing the enclosed chamber to be turned around more quickly for its next use. In another example embodiment, the catalyst medium may include glass beads or pellets which are coated with MnO₂, although other suitable media may also be used depending upon the given chemical solution that is to be used in the treatment cycle.

In some embodiments, both the dehumidification chamber 45 and the catalyzation chamber may be run in parallel, or the controller 42 may alternate back and forth between them. In still another implementation, the controller 42 could prompt a user to switch out a desiccation pod for a catalyzation pod after desiccation is complete, such that only a single pod chamber is required within the fogging injection station 32. In this regard, the pods may be identifiable by type (e.g., via an RFID circuit, bar or QR code, etc.), which the controller 42 recognizes and then only allows use during the appropriate treatment phase, for example.

In still other embodiments, a combination pod could be used within the dehumidification chamber which includes both desiccation and catalyst media. For example, a pod may include a desiccant in a range of 50 to 75%, and a catalyst in a range of 25% to 50%. In some embodiments, the desiccant and catalyst media could be distributed throughout the pod (i.e., mixed together), or the pod may be a two-stage pod with a breathable membrane that keeps the two media separated within the pod yet still allows air flow through the pod.

Once the catalyzation phase is concluded (Block 111), the treatment cycle is complete (Block 112). It should be noted that various approaches may be used for determining when the pre-treatment, injection, pulse, evacuation, and/or catalyzation phases are complete. For example, this may be based on a set time for the given phase, a set number of cycles (e.g., pulses) to be performed during the phase, or based upon sensor measurements (e.g., humidity, H₂O₂ concentration, etc.) indicating that a target parameter for the particular phase or cycle has been reached.

It should also be noted that one or more of the above-described pre-treatment, intermittent dehumidification/desiccation, and catalyzation phases may be used with other fogging or injection devices besides the fogging injection station 32. For example, it may be used with a stand-alone fogging device, or a plurality of such fogging devices working in parallel or on a coordinated schedule, such as those described in the above-noted '668 patent.

The above-described intermittent dehumidification/desiccation when used at the beginning of the treatment cycle advantageously helps to more rapidly increase the PPM of the active ingredient (e.g., H₂O₂) in the treatment space, yet without significantly affecting the environmental relative humidity (RH). The system may inject a small amount of solution to bring up the RH and H₂O₂ ppm in the space. The intermittent dehumidification/desiccation is performed to extract humidity and bring the RH back down, which also helps raise H₂O₂ ppm, and subsequent intermittent dehumidification/desiccation cycles may be performed as desired. More particularly, even when performed multiple times this may result in a relatively small rise in RH, but it may provide a significantly higher increase in the H₂O₂ ppm level. After multiple cycles, the regular injection and/or pulse phases may commence as discussed above, yet at the start of the injection phase the RH may advantageously be close to or only slightly higher than if the pre-treat intermittent dehumidification/desiccation had not happened.

In some embodiments, an intermittent dehumidification/desiccation phase(s) may be performed at a later time in the treatment cycle in addition to (or instead of) during the pre-treatment phase. This may be particularly beneficial for relatively long or extended treatment cycles, for example. More particularly, bio-decontamination agents such as H₂O₂ will degrade for multiple reasons during the decontamination cycle (e.g., it may be catalyzed due to organics, surface type, or just breakdown over time). Thus, while a treatment solution will have a particular starting concentration (e.g., 7%), once atomized into a treatment space this concentration will be reduced over time. At some point during the active dwell or pulse phase of the treatment cycle, an intermittent dehumidification/desiccation phase may be performed to extract the space down to a lower RH (e.g., 70%, although other values may be used in different embodiments), but then reinjection may occur to bring the space back to the desired concentration. This may provide several technical advantages. First, this may provide the above-described effect of increasing the H₂O₂ ppm level while decreasing the RH. Moreover, if the solution in the system has been reduced in terms of its active concentration (e.g., 7% down to 4%), this has the effect of “drying out” the enclosure slightly and then reinjecting new higher concentration (here 7%) solution. In an example implementation, for extended cycles of two hours to multiple days, it may be desirable to add intermittent dehumidification/desiccation phases at certain intervals, e.g., once an hour, although other timing and intervals may be used in different embodiments. This advantageously helps keep the H₂O₂ at the proper concentration within the treatment space, while also maintaining “fresh” solution in the space throughout the entire treatment cycle.

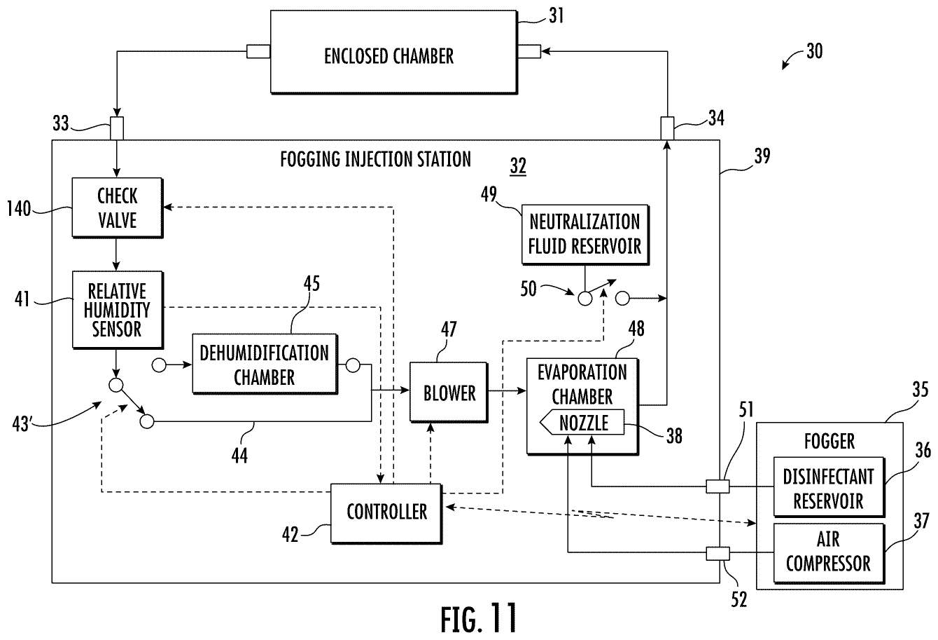

Turning now to FIG. 11, in accordance with another example embodiment the fogging injection station 32 may include a check valve 140 instead of (or in addition to) the above-noted pressure release valve 40. The check valve 140 is positioned upstream of the blower 47 in the airflow path and is coupled to the controller 42. The controller 42 controls the check valve 140 and blower 47 so that air is introduced into the enclosed chamber 31 but is not allowed to exit. In the case of a biological safety cabinet with integrated rubber gloves (also known as glove boxes or isolation glove boxes), this creates a positive pressure to inflate the gloves outward from the cabinet. That is, the gloves are inflated to remove creases and expose all interior surfaces so that they can be exposed to the atomized treatment fluid during the treatment cycle. Once the gloves are properly inflated, the check valve 140 may be opened by the controller 42 when the treatment cycle is performed, as discussed further above. By way of example, the controller 42 may be responsive to user input from the control panel 60 (or a control panel associated with the enclosed chamber 31 in some embodiments) as an indication when the gloves are fully inflated. This could also be determined based upon a pressure level detected by the controller 42 via a pressure sensor (not shown) based upon a threshold pressure, in some embodiments.

Also in the illustrated example, the atomizing nozzle 38 is oriented within the evaporation chamber 48 to face upstream in the airflow path. In some applications, this may help to maintain a dryer treatment cycle, as it may provide the atomized solution more room to “plume” out or disperse into the airflow before condensing. Here again, this could be done without an evaporation chamber 48 in some embodiments, i.e., the nozzle 38 may be introduced directly into the airflow path without a separate evaporation chamber. Moreover, it should also be noted that other orientations of the nozzle 38 may be used in different embodiments. For example, the nozzle 38 may be oriented at any angle from zero (pointing directly downstream with the airflow) to one hundred eighty (pointing directly upstream into the airflow).

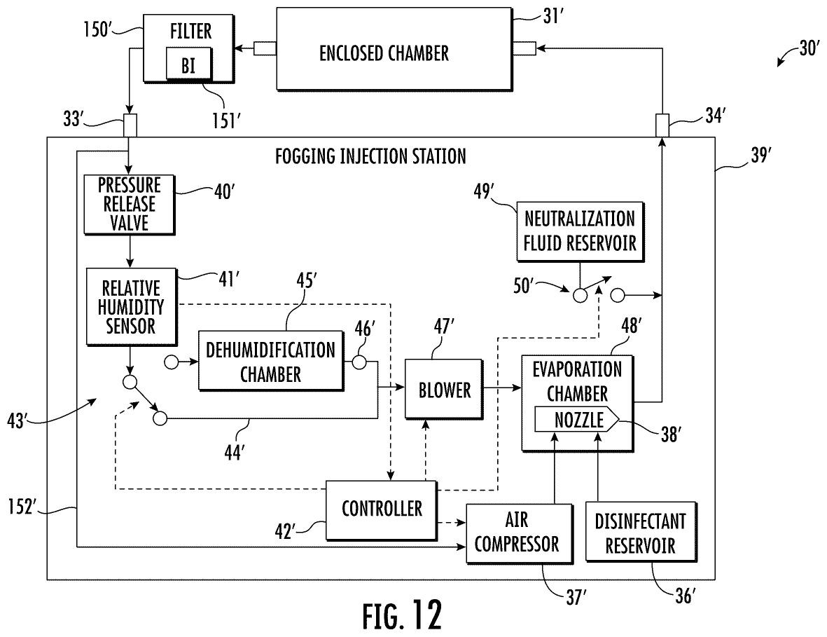

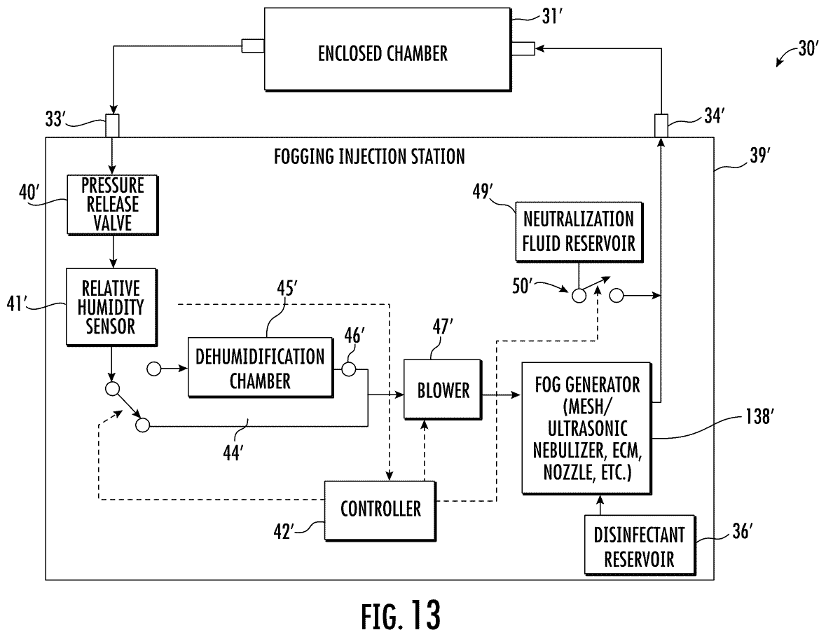

Turning now to FIG. 12, in another example embodiment of the system 30′ a filter interconnect device 150′ may be connected between the outlet of the enclosed chamber 31′ and the inlet 33′ of the fogging injection station 32′. The filter interconnect device 150′ provides an inline filter (e.g., a HEPA filter) so that the fogging injection station 32′ may be disconnected from the treatment circuit without releasing any pathogens from the enclosed chamber 31′. More particularly, this may be particularly advantageous when treating enclosed chambers 31′ that do not have an integrated HEPA filter, as disconnecting from the enclosed chamber 31′ before a treatment cycle is completed could otherwise release pathogens from the enclosed chamber.

In an example embodiment, the filter interconnect device 150′ may include a housing with inlets/outlets to connect to the enclosed chamber 31′ and the fogging injection station 32′, respectively, in which a filter cartridge may be inserted and removed when it is time for a replacement. For example, this may take the form of a threaded cylindrical filter cartridge that screws into the filter interconnect device 150′, although other form factors may be used in different embodiments. In some embodiments, an RFID tag may be included in the filter cartridge to help ensure the proper cartridge type is being used, and to determine when it is time to change the filter cartridge.

Moreover, in some embodiments the filter interconnect device 150′ may be configured to receive a replaceable biological indicator(s) (BI) 151′ (e.g., a Geobacillus stearothermophilus test strip) for efficacy testing, as will be appreciated by those skilled in the art. The BI 151′ may be positioned downstream from the filter cartridge in the airflow path. This configuration advantageously allows for convenient access to the BI 151′ to verify treatment efficacy. Clips or other suitable holders may be used for securing the BI 151′ within the filter interconnect device 150′.

Still another advantageous feature of the present embodiment is that a compressor air intake 152′ for the air compressor 37′ is pulling from the return piping in the airflow path of the treatment loop. That is, the illustrated system 30′ of FIG. 12 is a closed loop system, in which no external air is introduced into the airflow path by the air compressor 37′, which may be desired in some treatment applications.

Generally speaking, the systems set forth herein may incorporate various types of disinfectant fog or mist generators. As used herein, a “fog generator” is a device that causes fine particles or droplets of a treatment fluid to be introduced into the air flow path in a form such as an aerosol, vapor, fog, mist, or atomized fluid. Examples of fog generators 138′ include atomizing nozzles, as discussed above, as well as nebulizers and mechanical vaporizers/aerosolizers (e.g., vibrating membranes, composite screen diffusion, etc.). In some embodiments, more than one type of fog generator may be used introduce disinfectant solution mist or fog into the airflow path. An advantage of using a nebulizer(s) instead of an atomizing nozzle is that it allows the air compressor 37′ to be omitted. This configuration may be advantageous for smaller enclosed chambers, such as cleanroom pass-through chambers, for example, although nebulizers may be used with larger enclosed chambers as well. As with the atomizing nozzle 38′ described above, nebulizers may be oriented in different directions so that the atomized solution is directed at different angles relative to the direction of airflow. Moreover, multiple nebulizers may be located at different positions along the airflow path and orientations, either within the evaporation chamber 48′ or elsewhere in embodiments where an evaporation chamber is not included.

Various types of nebulizers may be used in different embodiments. In one example implementation, a mesh nebulizer(s) may be used, in which pressurized fluid is delivered through a mesh or screen with small holes that creates consistent vapor/atomized particles or droplets in the air flow path. Mesh materials may include plastic, corrosion-resistant metals, etc., and may have mesh sizes of <500 microns, and more preferably in a range of 10 microns or less, for example.

Another type of nebulizer which may be used is an ultrasonic nebulizer. An ultrasonic nebulizer uses high-frequency sound waves from a transducer to turn liquids into aerosolized fog particles or droplets. By way of example, ultrasonic piezo elements, which are also called atomizing transducers or piezo atomizers, work by creating a mechanical (vibration) response to an electrical input. When an electrical charge or input (e.g., an AC voltage) is applied to the atomizing transducer, the piezo element generates ultrasonic vibrations.

Another related type of nebulizer which may be used is a vibrating mesh nebulizer. Vibrating mesh nebulizers or atomizers leverage the surface tension of a liquid to extrude it into a cloud of droplets through contact with a vibrating microperforated membrane or mesh. The vibration of the membrane on the surface of the liquid produces fine particles or droplets, the size of which depends on the size of the mesh holes. Because the atomization occurs through ultrasonic vibration of the membrane (e.g., at a vibratory frequency above 40 kHz), the liquid is not altered nor heated in the process. Again, this is also an advantage shared with atomizing nozzles and the other types of nebulizers mentioned above which all perform fog generation at room temperature without added heat. By contrast, heat-based vaporization systems which heat disinfection solutions to their boiling point to create vapor may pose additional safety risks to operations, and may consume more energy than some room temperature fog generator alternatives, for example.

Another type of example fog generator 138′ incorporates an evaporative cooling medium (ECM) in conjunction with the blower 47′, to provide room temperature or “cold” evaporation of disinfectant droplets or particles into the airflow path. An evaporative cooling medium, e.g., in the form of pads or panels, is positioned in the airflow path (e.g., in an evaporation chamber 48′ or directly in line) to provide a relatively large surface area by which heat is removed from air blown through/across the media, as the solution is evaporated into the air flow path. By way of example, the evaporative cooling media may be made from natural or synthetic fibers (wood wool, cellulose paper, aspen fibers, etc.), that absorb disinfectant fluid dripped or otherwise introduced to the media from the disinfectant reservoir 36′ (e.g., via a pump, gravity feed, etc.). Various geometries of pads or panels may be used, including rectangular or other shapes (cylindrical, etc.) appropriate for the evaporation chamber 48′ or other location of the ECM.

In some embodiments, a filter medium may similarly be used ECM for evaporation of droplets or particles. By way of example, the filter interconnect device 150′ shown in FIG. 12 in some embodiments may be positioned upstream of the enclosure 31′ (as opposed to downstream as shown in FIG. 12), and disinfectant fluid introduced to the filter medium (e.g., via a pump, gravity feed, etc.). Here again, the force of the air created by the blower 47′ may cause liquid on the filter media to be evaporated into particles or drops that proceed along the airflow path into the enclosed chamber 31′. In some embodiments, the filter medium may be located within the fogging injection station 39, as discussed with the other ECMs noted above, or at the enclosed chamber 31′, for example.

It should also be noted that one or more of the above-described features may be used with other fogging devices, such as those described in the above-noted '668 patent. Moreover, while a given feature of the fogging injection stations 32, 32′ may have been described in the context of a particular configuration above, such features may be applicable to and utilized in other configurations as well.

Moreover, it should also be noted that the above-described components may be incorporated within a same, common housing, or various components may be separate from the housing (e.g., in separate housings). For example, in some embodiments a separate fluid reservoir, compressor, dehumidification chamber, etc., may be remotely located from a fogging injection station/fogger, and/or shared by different fogging injection stations/foggers. While not shown, the above-described fogging injection stations/foggers may be controlled remotely (e.g., via a wired or wireless connection to a computer or mobile communications device), or by a local human machine interface (HMI), such as a touch screen. In some embodiments, the HMI may be carried on a same housing with the fogging injection station/fogger, but in other embodiments the HMI may be associated with a chamber or cabinet to which the fogging injection station/fogger is connected. That is, the HMI used to operate the chamber/cabinet may also be configured to interface with the fogging injection station/fogger to control its operation as well.

Many modifications and other embodiments will come to the mind of one skilled in the art having the benefit of the teachings presented in the foregoing descriptions and the associated drawings. Therefore, it is understood that the foregoing is not to be limited to the example embodiments, and that modifications and other embodiments are intended to be included within the scope of the appended claims.

1. A system for disinfecting an enclosed chamber comprising:
a housing;
an inlet and an outlet carried by the housing to be connected in an airflow path with the enclosed chamber;
a blower carried by the housing and connected to the airflow path between the inlet and outlet and configured to circulate air through the airflow path;
a disinfectant fog generator configured to introduce a disinfectant fog into the airflow path at room temperature from a disinfectant fluid; and
a controller configured to operate the disinfectant fog generator and a dehumidifier coupled to the airflow path during a treatment phase.

2. The system of claim 1 wherein the disinfectant fog generator comprises an ultrasonic nebulizer.

3. The system of claim 1 wherein the disinfectant fog generator comprises a vibrating mesh nebulizer.

4. The system of claim 1 wherein the disinfectant fog generator comprises an absorbent medium in the airflow path downstream from the blower, and a disinfectant fluid dispenser configured to dispense fluid to the absorbent medium responsive to the controller.

5. The system of claim 1 wherein the disinfectant fog generator comprises an atomizing nozzle.

6. The system of claim 1 wherein the controller is configured to operate the blower non-continuously during the treatment phase.

7. The system of claim 1 wherein the controller is configured to operate the disinfectant fog generator continuously during a first portion of the treatment phase, and non-continuously during a second portion of the treatment phase.

8. The system of claim 1 wherein the dehumidifier comprises a desiccation cartridge; and further comprising an airflow valve downstream from the inlet and configured to switch the airflow through the desiccation cartridge responsive to the controller.

9. The system of claim 1 wherein the dehumidifier comprises an evaporator.

10. A system for treating an enclosed chamber comprising:
a housing;
an inlet and an outlet carried by the housing to be connected in an airflow path with the enclosed chamber;
a blower carried by the housing and connected to the airflow path between the inlet and outlet and configured to circulate air through the airflow path;
a fog generator configured to introduce a fog into the airflow path at room temperature from a treatment fluid; and
a controller configured to operate the fog generator and a dehumidifier coupled to the airflow path during a treatment phase.

11. The system of claim 10 wherein the fog generator comprises an ultrasonic nebulizer.

12. The system of claim 10 wherein the fog generator comprises a vibrating mesh nebulizer.

13. The system of claim 10 wherein the fog generator comprises an absorbent medium in the airflow path downstream from the blower, and a fluid dispenser configured to dispense fluid to the absorbent medium responsive to the controller.

14. The system of claim 10 wherein the fog generator comprises an atomizing nozzle.

15. The system of claim 10 wherein the controller is configured to operate the blower non-continuously during the treatment phase.

16. The system of claim 10 wherein the controller is configured to operate the fog generator continuously during a first portion of the treatment phase, and non-continuously during a second portion of the treatment phase.

17. A method for disinfecting an enclosed chamber comprising:
connecting an inlet and an outlet of a fluid injection station in a closed airflow path with the enclosed chamber, the fluid injection station comprising a housing carrying the inlet and outlet, a blower carried by the housing and connected to the airflow path between the inlet and outlet, and a disinfectant fog generator coupled to the airflow path;
operating the blower to circulate air through the airflow path during a treatment phase;
operating the disinfectant fog generator to introduce a disinfectant fog into the airflow path at room temperature from a disinfectant fluid during the treatment phase; and
operating a dehumidifier coupled to the airflow path during the treatment phase.

18. The method of claim 17 wherein the disinfectant fog generator comprises an ultrasonic nebulizer.

19. The method of claim 17 wherein the disinfectant fog generator comprises a vibrating mesh nebulizer.

20. The method of claim 17 wherein the disinfectant fog generator comprises an absorbent medium in the airflow path downstream from the blower, and a disinfectant fluid dispenser configured to dispense fluid to the absorbent medium.

21. The method of claim 17 wherein the disinfectant fog generator comprises an atomizing nozzle.

22. The method of claim 17 wherein operating the blower comprises operating the blower non-continuously during the treatment phase.

23. The method of claim 17 wherein operating the disinfectant fog generator comprises operating the disinfectant fog generator continuously during a first portion of the treatment phase, and non-continuously during a second portion of the treatment phase.
